(19) United States
(12) Patent Application Publication
(10) Pub. No.: US 2009/0231587 A1
(43) Pub. Date: Sep. 17, 2009

(54) DEVICE FOR IMAGING AN INTERIOR OF A TURBID MEDIUM

(75) Inventors: Levinus Pieter Bakker, Eindhoven (NL); Martinus Bernardus Van Der Mark, Eindhoven (NL); Michael Cornelis Van Beek, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/513,686

(22) PCT Filed: Nov. 13, 2007
(86) PCT No.: PCT/IB07/54601

§ 371 Date: May 6, 2009

(30) Foreign Application Priority Data
Nov. 17, 2006 (EP) 06124352.3

(51) Int. Cl. A61B 5/00

(52) U.S. Cl. 356/432

(57) ABSTRACT

The invention relates to a device (1) for imaging an interior of a turbid medium (55) comprising a receptacle (20) with the receptacle (20) comprising a measurement volume (15) for receiving the turbid medium (55). The device (1) is adapted such that the inner surface of the receptacle (20), including at least part of the optical channels (70) is covered with a layer (80).

### FIELD OF THE INVENTION

The invention relates to a device for imaging an interior of a turbid medium comprising a receptacle with the receptacle comprising a measurement volume for receiving the turbid medium and with the receptacle comprising optical channels for optically coupling a light source to the measurement volume. The invention also relates to a medical image acquisition device comprising the device.

### BACKGROUND OF THE INVENTION

An embodiment of a device of this kind is known from U.S. Pat. No. 6,327,488 B1. The known device can be used for imaging an interior of a turbid medium, such as biological tissues. In medical diagnostics the device may for example be used for imaging an interior of a female breast. The measurement volume of the device receives a turbid medium, such as a breast. The measurement volume may be bound by a holder having only one open side, with the open side being bound by an edge portion. This edge portion may be provided with an elastically deformable sealing ring. Such a holder is known from U.S. Pat. No. 6,480,281 B1. Light is applied to the turbid medium by irradiating the turbid medium from a position that is successively chosen from a number of positions. Light emanating from the measurement volume via further positions selected from the number of positions is detected by a detector unit and is used to derive an image of the interior of the turbid medium by reconstruction of the measurement.

In the above described example of optical mammography both the measurement of the light emanating from the measurement volume via further positions and the reconstruction thereof are greatly enhanced by immersing the female breast in a liquid that matches the average optical properties of the breast, a so-called adaptation or matching liquid. Such use of an adaptation fluid results in a counteraction of boundary effects stemming from the optical coupling of the turbid medium with its surroundings. Typically a fluid is used that matches the average properties of the average female breast.

### SUMMARY OF THE INVENTION

It is a drawback of the known device that the cleaning of the receptacle after use thereof is rather time consuming because of the presence of the optical channels. Moreover said optical channels are easily damaged. It is an object of the invention to adapt the device such that it is more easy to clean and where the risk of damaging the optical channels is considerably reduced. According to the invention this object is realized in that at least part of the inner surface of the receptacle, including at least part of the optical channels is covered with a layer.

By covering the surface of the receptacle that faces the measurement volume and at least one ends of the optical channels comprised in the receptacle with a layer of material, a surface is created that protects both the inner surface of the receptacle and the optical channels.

Preferably, the applied layer forms a continuous surface on the inner side of the receptacle.

Such surface protects the covered optical channels from damage and is easy to clean.

In an advantageous embodiment the optical properties of the layer applied resemble the optical properties of the average breast and, if present, the matching fluid.

Such layer decreases the measurement noise, since it increases the beam footprint at the surface between the receptacle, or up, and the adapting or matching fluid. surface. As a result of this, fluctuations of the fluid near the source and detector fibers are averaged out more.

It is advantageous to choose the optical properties of the layer such that the optical properties of the layer are similar to the optical properties of the turbid medium. ‘Optical properties similar to those of the turbid medium’ also covers optical properties that are averaged over a group of turbid mediums that may be imaged using the device.

Furthermore, the layer may be used to diffuse light exiting from the ends of covered optical channels and entering the measurement volume. Diffuse light has the advantage of being safer for people working with the device. If the device is used in medical diagnostics for, for instance, the imaging of a female breast, these people include patients who may look into the measurement volume before and after a breast is accommodated in the measurement volume. If the layer is used to diffuse light, the optical properties of the layer must be chosen such that the layer is sufficiently transparent for light exiting the end of a covered optical channel in a direction substantially perpendicular to the layer and entering the restricted measurement volume, so that a sufficient amount of light enters the restricted measurement volume. However, at the same time the optical properties of the layer must be chosen such that the layer is sufficiently absorbent for light exiting the end of a covered optical channel and traveling through the layer without entering the measurement volume so that only an insignificant amount of light might reach the end of a neighboring optical channel.

This can preferably be accomplished with materials that have either a high scattering coefficient or a high absorption coefficient or both. In an alternative preferred embodiment the material of the layer has been structured in such a way that it refracts or diffracts the light in the direction substantially perpendicular to the layer.

As materials with a high scattering coefficient scattering plastics are preferably use, such as for instance polyoxymethylene, polyamide, or a host material with scattering particles as for instance epoxy with scattering particles as for instance TiO₂, gas bubbles or glass spheres.

Examples of materials with a high absorption coefficient are absorption glass, welders' glass, epoxy mixed with a dye or a transparent material mixed with a dye.

Examples of materials with both a high scattering and a high absorption coefficient are epoxy mixed with a dye and scattering particles at an appropriate concentration.

The optical properties of these materials are such that the layer is sufficiently transparent for light exiting the end of a covered optical channel in a direction substantially perpendicular to the layer and entering the restricted measurement volume.

According to the invention the medical image acquisition device comprises the device according to any of the previous embodiments. If, for instance, the device is used to image an interior of a female breast, as is done in medical diagnostics, the device would benefit from any of the previous embodiments.

These and other aspects of the invention will be further elucidated and described with reference to the drawings, in which:

### BRIEF DESCRIPTION OF THE DRAWINGS

FIG. 1 schematically shows an embodiment of a device for performing measurements on a turbid medium,

FIG. 2 schematically shows a receptacle of which the inner surface, including the optical channels, is covered with a layer,

FIG. 3 schematically shows an embodiment of a medical image acquisition device according to the invention.

### DETAILED DESCRIPTION OF EMBODIMENTS

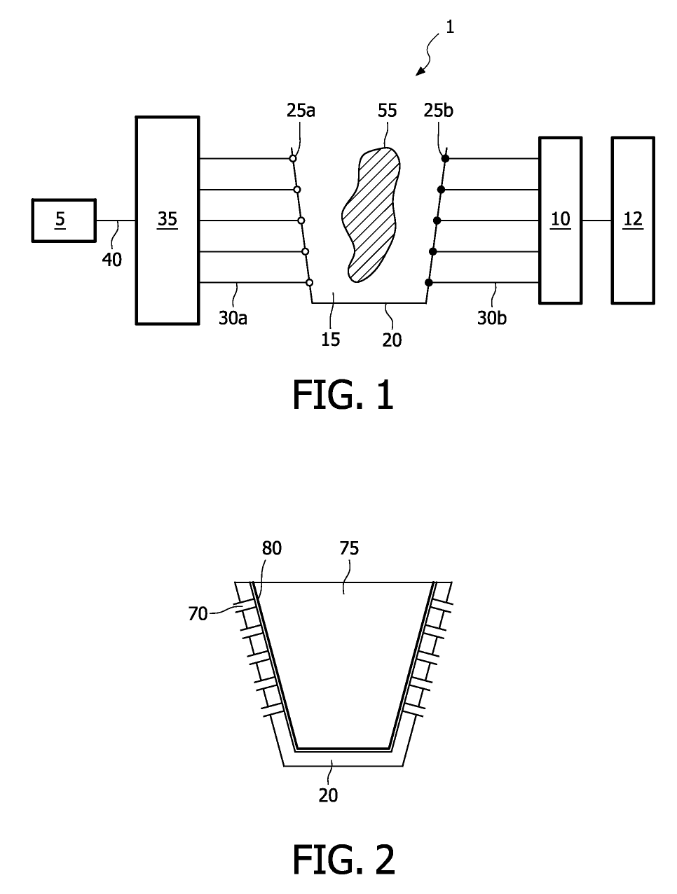

FIG. 1 schematically shows an embodiment of a device for imaging an interior of a turbid medium. The device 1 includes a light source 5, a photodetector unit 10, an image reconstruction unit 12 for reconstructing an image of an interior of the turbid medium 55 based on light detected using the photodetector unit 10, a measurement volume 15 bound by a receptacle 20, said receptacle 20 comprising a plurality of entrance positions for light 25a and a plurality of exit positions for light 25b, and light guides 30a and 30b coupled to said entrance and exit positions. The device 1 further includes a selection unit 35 for coupling the light source 5 to a number of selected entrance positions for light 25a in the receptacle 20. The light source 5 is coupled to the selection unit 35 using input light guides 40. For the sake of clarity, entrance positions for light 25a and exit positions for light 25b have been positioned at opposite sides of the receptacle 20. In reality, however, both types of positions may be spread around the measurement volume 15. A turbid medium 55 is accommodated in the measurement volume 15. The turbid medium 55 is then irradiated with light from the light source 5 from a plurality of positions by coupling the light source 5 using the selection unit 35 to successively selected entrance positions for light 25a. Light emanating from the measurement volume 15 is detected from a plurality of positions using exit positions for light 25b and using photodetector unit 10. The detected light is then used to derive an image of an interior of the turbid medium 55. This reconstruction process, which is based on, for example, an algebraic reconstruction technique or a finite element method finds the most likely solution to the inverse problem.

FIG. 2 schematically shows a receptacle 20. The receptacle 20 comprises optical channels 70 for optically coupling the light source 5 (see FIG. 1) to the measurement volume 15 which is enclosed by the receptacle 20. Also shown in FIG. 2 is an optical fiber 72 coupled to the receptacle 20. The inner side of the receptacle 20, including the optical channels, is coated with a thin layer of material 80 that preferably resembles the optical properties of the average breast and, if present, the matching fluid. In this way a continuous inside wall is created that protects the optical channels from damage and is easy to clean. Furthermore, this coating decreases the measurement noise, since it increases the beam footprint at the cup-fluid surface. As a result of this, fluctuations of the fluid near the source and detector fibers are averaged out more.

Applications of a continuous layer may be, for instance, to diffuse or absorb light exiting the optical channel 80 into the measurement volume 15. If the layer is used to diffuse light, the optical properties of the layer must be chosen such that the layer is sufficiently transparent for light exiting a covered optical channel 70 in a direction substantially perpendicular to the layer and entering the measurement volume 15, so that a sufficient amount of light enters the measurement volume 15. Polyoxymethylene is an example of the material that has the required optical properties. Alternatively, the layer may be made of a material such as welders' glass. In that case, light exiting a covered optical channel 70 will be less diffuse than if a material such as polyoxymethylene were used. However, a material such as welders' glass absorbs light more strongly than a material such as polyoxymethylene. So there is a range of materials with on the one end materials such as polyoxymethylene that diffuse light, but absorb light relatively weakly and materials such as welders' glass on the other end that basically do not diffuse light, but absorb light relatively strongly. Optimal conditions may be created by choosing the layer material and layer thickness.

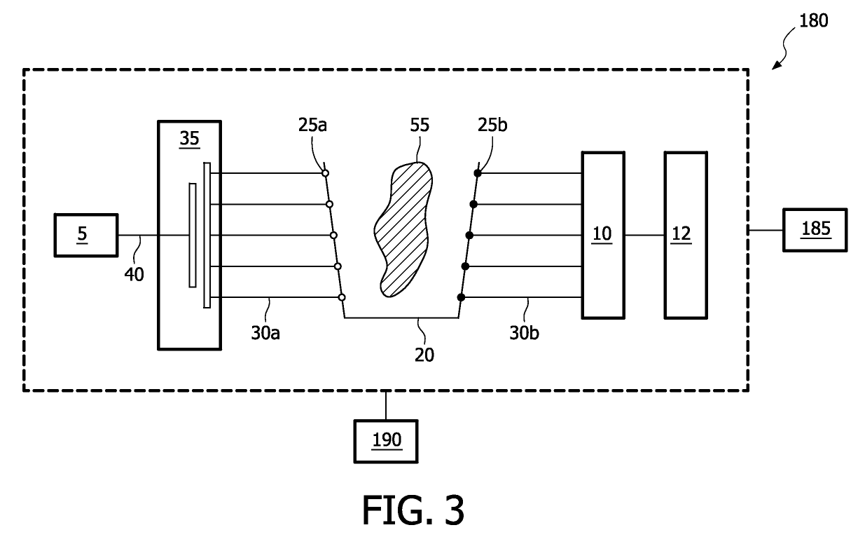

FIG. 3 shows embodiment of a medical image acquisition device according to the invention. The medical image acquisition device 180 comprises the device 1 discussed in FIG. 1 as indicated by the dashed square. In addition to the device 1 the medical image acquisition device 180 further comprises a screen 185 for displaying an image of an interior of the turbid medium 45 and an input interface 190, for instance, a keyboard enabling and operated to interact with the medical image acquisition device 180.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. The word “comprising” does not exclude the presence of elements or steps other than those listed in a claim. The word “a” or “an” preceding an element does not exclude the presence of a plurality of such elements. In the system claims enumerating several means, several of these means can be embodied by one and the same item of computer readable software or hardware. The mere fact that certain measures are recited in mutually different dependent claims does not indicate that a combination of these measures cannot be used to advantage.

1. A device (1) for imaging an interior of a turbid medium (55) comprising a receptacle (20) with the receptacle (20) comprising a measurement volume (15) for receiving the turbid medium (55) and with the receptacle (20) comprising optical channels (25) for optically coupling a light source (5) to the measurement volume (15) wherein at least part of the inner surface of the receptacle (20), including at least part of the optical channels (70) is covered with a layer (80).

2. A device (1) as claimed in claim 1, wherein the layer (80) comprises a continuous layer.

3. A device (1) as claimed in claim 1, wherein the layer (80) refracts or diffracts light passing through said layer in a direction substantially perpendicular to the layer.

3. A device (1) as claimed claim 1, wherein the layer (80) comprises a material with optical properties similar to the optical properties of the turbid medium (55).

4. A device (1) s claimed in claim 1, wherein the layer comprises a material with a high scattering coefficient.

5. A device (1) as claimed in claim 1, wherein the layer comprises polyoxymethylene or polyamide.

6. A device (1) as claimed in claim 1, wherein the layer comprises a host material with scattering particles.

7. A device (I) as claimed in claim 1, wherein the layer comprises a material with a high absorption coefficient.

8. A device (1) as claimed in claim 1, wherein the layer comprises absorption glass, welders' glass, epoxy mixed with a dye or a transparent material mixed with a dye.

9. A device (1) as claimed in claim 1, wherein the material of the layer has been structured in such a way that it refracts or diffracts the light in the direction substantially perpendicular to the layer.

10. A medical image acquisition device comprising the device (1) according to claim 1.
